(19) United States
(12) Patent Application Publication
(10) Pub. No.: US 2022/0068481 A1
(43) Pub. Date: Mar. 3, 2022

(54) METHODS AND SYSTEM FOR OBTAINING A PHYSIOLOGICAL MEASURE FROM A SUBJECT

(71) Applicant: KONINKLIJKE PHILIPS N.V., EINDHOVEN (NL)

(72) Inventors: Ralph Theodorus Hubertus MAESSEN, ROERMOND (NL); Marco BARAGONA, DELFT (NL); Kevin Daniel Seng Hung LAU, EINDHOVEN (NL)

(21) Appl. No.: 17/413,703

(22) PCT Filed: Dec. 19, 2019
(86) PCT No.: PCT/EP2019/086182

§ 371 Date: Jun. 14, 2021

Related U.S. Application Data
(60) Provisional application No. 62/782,418, filed on Dec. 20, 2018.

(51) Int. Cl. G16H 50/20; G16H 50/50; A61B 5/02; A61B 5/021; A61B 5/024; A61B 5/00

(52) U.S. Cl. CPC G16H 50/20; G16H 50/50; A61B 5/02028; A61B 5/742; A61B 5/021; A61B 5/024; A61B 5/02007

(57) ABSTRACT

The invention provides a method for obtaining a physiological measure from a subject, in particular a P-V loop. The method includes obtaining a numerical model of a cardiac system and obtaining, in a non-invasive manner, physiological data from the subject. The numerical model is then updated based on the physiological data. The physiological data is then provided to the updated numerical model and a physiological measure is derived based on an output of the updated numerical model, wherein the physiological measure includes a P-V loop.

### FIELD OF THE INVENTION

This invention relates to the field of physiological measurements, and more specifically to the field of using physiological measurements to model a physiological system.

### BACKGROUND OF THE INVENTION

The pumping function of the heart can be characterized by the ejection phase and the filling phase. During ejection, the muscles of the heart contract generating a force that ejects blood from the heart into the circulation. Conversely, during filling, the muscles of the heart relax enabling the refilling of blood from the circulation into the heart. Impairment of either the contraction or the relaxation of the heart muscles may result in systolic or diastolic heart failure.

As the direct measurement of the muscular contraction and relaxation is not typically possible in a clinical setting, surrogate measures are typically utilized to characterize heart function, such as hemodynamics and ventricular motion.

A key surrogate measure is the ventricular blood pressure-volume curve, as referred to as a P-V loop, due to its direct relationship with the forces generated and sustained by the heart during contraction, relaxation, myocardial energetics and so on. However, typically the ventricular pressure component of the P-V loop can only be measured invasively via catheterization. As this is a highly invasive procedure, such catheter-based ventricular pressure measurements are uncommon in clinical practice.

A variety of non-invasive estimates for ventricular pressure exist. Such estimates are typically population based, where empirical relationships are used to correlate pressure with non-invasively measured variables, such as cuff pressure and ultrasound measured stroke volume for end-systolic pressure estimation or Doppler tissue imaging for filling pressure estimation.

Due to the indirect nature of these estimates, the pressure in an individual patient can be over- or under-estimated with such techniques. Furthermore, these estimates focus solely upon systolic or diastolic pressure estimation; however, the progression of heart failure is increasingly viewed as a coupling of systolic and diastolic dysfunction.

There is therefore a need for a non-invasive method for estimating heart function during both contraction and relaxation of the heart.

### SUMMARY OF THE INVENTION

The invention is defined by the claims.

According to examples in accordance with an aspect of the invention, there is provided a method for obtaining a physiological measure from a subject, the method comprising:

obtaining a numerical model of a cardiac system;

obtaining, in a non-invasive manner, physiological data from the subject;

updating the numerical model based on the physiological data;

providing the physiological data to the updated numerical model; and

deriving a physiological measure based on an output of the updated numerical model, the physiological measure comprising a pressure-volume, P-V, loop.

The method provides for non-invasively deriving a P-V loop by way of a numerical model, which may be personalized to a user and the available physiological data, thereby increasing the accuracy of the derived physiological measure.

In this way, the measured data is provided to a user specific numerical simulation of a cardiac system before the final measure is derived. This avoids the typical requirement for invasive measurement, whilst overcoming accuracy issues present in current non-invasive alternatives.

Typically, the only way to derive a complete P-V loop for a patient is via the highly invasive use of catheters, which is typically not employed as part of standard routine. Current, non-invasive alternatives typically focus on deriving only a few points or part of a pressure-volume loop. Furthermore, these estimates are typically population-based, empirical relationships correlating pressure with the non-invasively measured variables.

Thus, the numerical model provides a means to accurately simulate the function of a cardiac system of a subject, thereby allowing for the simulation of cardiac functions in a personalized manner. Accordingly, the numerical model provides a non-invasive means of deriving such metrics including the pressure-volume loop without sacrificing the accuracy of the final measure.

The non-invasive estimation of cardiac function, such as a ventricular pressure-volume relation, provides for an assessment of heart function in a variety of different clinical settings. Further, the method may be employed regularly without incurring the risks associated with invasive procedures.

In an embodiment, the physiological data comprises one or more of:

ultrasound data;

electrocardiogram data; and

a blood pressure.

These data types allow for a large range of physiological measures to be derived.

In a further embodiment, the ultrasound data comprises one or more of:

2D ultrasound image data;

3D ultrasound image data; and

Doppler ultrasound data.

The availability of a given type of ultrasound data may be limited in certain clinical situations; however, any available type may be used to generate a useful measure.

In an embodiment, the blood pressure comprises one or more of:

an arterial pressure waveform; and

a cuff pressure value.

The availability of a given type of pressure data may be limited in certain clinical situations; however, any available type may be used to generate a useful measure.

In an arrangement, the numerical model is based on:

a pressure-volume relationship;

a stiffness of the cardiac system;

conservation of energy;

conservation of mass; and

conservation of momentum.

For example, by adhering to such physical conservation laws, the accuracy of the numerical model may be increased, thereby increasing the accuracy of the final measure.

In an embodiment, the cardiac system comprises a left ventricle and systemic arteries.

For example, the cardiac system may include the left heart and the systemic arteries.

In an embodiment, the cardiac system comprises a heart.

In an arrangement, the updating of the numerical model comprises:

identifying a model parameter based on the physiological data; and

providing the model parameter to the numerical model.

In this way, user specific data may be utilized to set the parameters for the numerical model, thereby personalizing the numerical model to an individual user.

In a further embodiment, the model parameter comprises one or more of:

a systemic circulation parameter;

a filling parameter;

an ejection parameter; and

a stiffness parameter.

In an arrangement, the physiological measure comprises a pressure-strain relationship.

In an embodiment, the physiological measure comprises a fluid responsiveness.

In an embodiment, the method further comprises displaying the physiological measure to a user.

According to examples in accordance with an aspect of the invention, there is provided a computer program comprising computer program code means which is adapted, when said computer program is run on a computer, to implement the method described above.

According to examples in accordance with an aspect of the invention, there is provided a processing unit for obtaining a physiological measure from a subject, wherein the processing unit is adapted to:

obtain a numerical model of a cardiac system;

update the numerical model based on the physiological data;

provide the physiological data to the updated numerical model; and

derive a physiological measure based on an output of the updated numerical model, wherein the physiological measure comprises a pressure-volume, P-V, loop.

According to examples in accordance with an aspect of the invention, there is provided a system for obtaining a physiological measure from a subject, the system comprising:

a physiological sensor adapted to obtain physiological data from the subject in a non-invasive manner, wherein the physiological sensor comprises one or more of:

an ultrasound transducer, wherein the physiological data comprises ultrasound data;

an electrocardiogram sensor, wherein the physiological data comprises electrocardiogram data; and

a blood pressure measurement device, wherein the physiological data comprises blood pressure data; and

a processor as described above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows an ultrasound diagnostic imaging system to explain the general operation;

FIG. 2 shows a method of the invention;

FIG. 3 shows a schematic representation of a numerical model;

FIG. 4 shows a graph illustrating pressure-volume loops;

FIG. 5 shows a graph illustrating pressure-volume relations;

FIG. 6 shows a graph illustrating fluid responsiveness; and

FIG. 7 shows a graph illustrating a ventriculo-arterial coupling.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a method for obtaining a physiological measure from a subject, in particular a P-V loop. The method includes obtaining a numerical model of a cardiac system and obtaining, in a non-invasive manner, physiological data from the subject. The numerical model is then updated based on the physiological data. The physiological data is then provided to the updated numerical model and a physiological measure is derived based on an output of the updated numerical model, wherein the physiological measure includes a P-V loop.

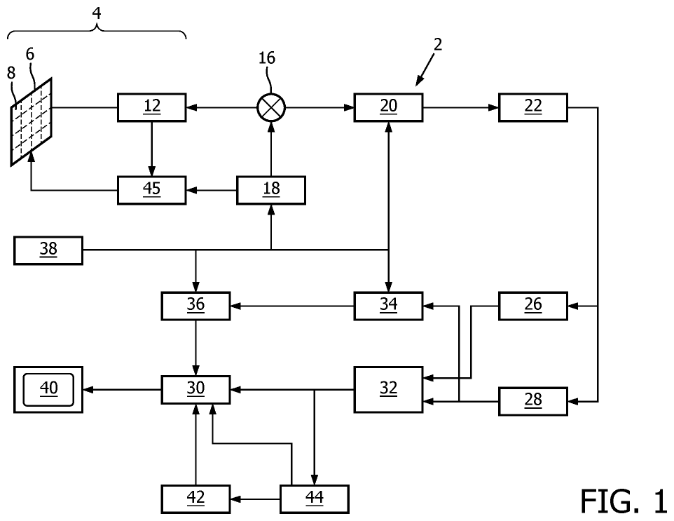

The general operation of an exemplary ultrasound system will first be described, with reference to FIG. 1, and with emphasis on the signal processing function of the system since this invention relates to the processing of the signals measured by the transducer array.

The system comprises an array transducer probe 4 which has a transducer array 6 for transmitting ultrasound waves and receiving echo information. The transducer array 6 may comprise CMUT transducers; piezoelectric transducers, formed of materials such as PZT or PVDF; or any other suitable transducer technology. In this example, the transducer array 6 is a two-dimensional array of transducers 8 capable of scanning either a 2D plane or a three dimensional volume of a region of interest. In another example, the transducer array may be a 1D array.

The transducer array 6 is coupled to a microbeamformer 12 which controls reception of signals by the transducer elements. Microbeamformers are capable of at least partial beamforming of the signals received by sub-arrays, generally referred to as “groups” or “patches”, of transducers as described in U.S. Pat. No. 5,997,479 (Savord et al.), U.S. Pat. No. 6,013,032 (Savord), and U.S. Pat. No. 6,623,432 (Powers et al.).

It should be noted that the microbeamformer is entirely optional. Further, the system includes a transmit/receive (T/R) switch 16, which the microbeamformer 12 can be coupled to and which switches the array between transmission and reception modes, and protects the main beamformer 20 from high energy transmit signals in the case where a microbeamformer is not used and the transducer array is operated directly by the main system beamformer. The transmission of ultrasound beams from the transducer array 6 is directed by a transducer controller 18 coupled to the microbeamformer by the T/R switch 16 and a main transmission beamformer (not shown), which can receive input from the user's operation of the user interface or control panel 38. The controller 18 can include transmission circuitry arranged to drive the transducer elements of the array 6 (either directly or via a microbeamformer) during the transmission mode.

In a typical line-by-line imaging sequence, the beamforming system within the probe may operate as follows. During transmission, the beamformer (which may be the microbeamformer or the main system beamformer depending upon the implementation) activates the transducer array, or a sub-aperture of the transducer array. The sub-aperture may be a one dimensional line of transducers or a two dimensional patch of transducers within the larger array. In transmit mode, the focusing and steering of the ultrasound beam generated by the array, or a sub-aperture of the array, are controlled as described below.

Upon receiving the backscattered echo signals from the subject, the received signals undergo receive beamforming (as described below), in order to align the received signals, and, in the case where a sub-aperture is being used, the sub-aperture is then shifted, for example by one transducer element. The shifted sub-aperture is then activated and the process repeated until all of the transducer elements of the transducer array have been activated.

For each line (or sub-aperture), the total received signal, used to form an associated line of the final ultrasound image, will be a sum of the voltage signals measured by the transducer elements of the given sub-aperture during the receive period. The resulting line signals, following the beamforming process below, are typically referred to as radio frequency (RF) data. Each line signal (RF data set) generated by the various sub-apertures then undergoes additional processing to generate the lines of the final ultrasound image. The change in amplitude of the line signal with time will contribute to the change in brightness of the ultrasound image with depth, wherein a high amplitude peak will correspond to a bright pixel (or collection of pixels) in the final image. A peak appearing near the beginning of the line signal will represent an echo from a shallow structure, whereas peaks appearing progressively later in the line signal will represent echoes from structures at increasing depths within the subject.

One of the functions controlled by the transducer controller 18 is the direction in which beams are steered and focused. Beams may be steered straight ahead from (orthogonal to) the transducer array, or at different angles for a wider field of view. The steering and focusing of the transmit beam may be controlled as a function of transducer element actuation time.

Two methods can be distinguished in general ultrasound data acquisition: plane wave imaging and “beam steered” imaging. The two methods are distinguished by a presence of the beamforming in the transmission (“beam steered” imaging) and/or reception modes (plane wave imaging and “beam steered” imaging).

Looking first to the focusing function, by activating all of the transducer elements at the same time, the transducer array generates a plane wave that diverges as it travels through the subject. In this case, the beam of ultrasonic waves remains unfocused. By introducing a position dependent time delay to the activation of the transducers, it is possible to cause the wave front of the beam to converge at a desired point, referred to as the focal zone. The focal zone is defined as the point at which the lateral beam width is less than half the transmit beam width. In this way, the lateral resolution of the final ultrasound image is improved.

For example, if the time delay causes the transducer elements to activate in a series, beginning with the outermost elements and finishing at the central element(s) of the transducer array, a focal zone would be formed at a given distance away from the probe, in line with the central element(s). The distance of the focal zone from the probe will vary depending on the time delay between each subsequent round of transducer element activations. After the beam passes the focal zone, it will begin to diverge, forming the far field imaging region. It should be noted that for focal zones located close to the transducer array, the ultrasound beam will diverge quickly in the far field leading to beam width artifacts in the final image. Typically, the near field, located between the transducer array and the focal zone, shows little detail due to the large overlap in ultrasound beams. Thus, varying the location of the focal zone can lead to significant changes in the quality of the final image.

It should be noted that, in transmit mode, only one focus may be defined unless the ultrasound image is divided into multiple focal zones (each of which may have a different transmit focus).

In addition, upon receiving the echo signals from within the subject, it is possible to perform the inverse of the above described process in order to perform receive focusing. In other words, the incoming signals may be received by the transducer elements and subject to an electronic time delay before being passed into the system for signal processing. The simplest example of this is referred to as delay-and-sum beamforming. It is possible to dynamically adjust the receive focusing of the transducer array as a function of time.

Looking now to the function of beam steering, through the correct application of time delays to the transducer elements it is possible to impart a desired angle on the ultrasound beam as it leaves the transducer array. For example, by activating a transducer on a first side of the transducer array followed by the remaining transducers in a sequence ending at the opposite side of the array, the wave front of the beam will be angled toward the second side. The size of the steering angle relative to the normal of the transducer array is dependent on the size of the time delay between subsequent transducer element activations.

Further, it is possible to focus a steered beam, wherein the total time delay applied to each transducer element is a sum of both the focusing and steering time delays. In this case, the transducer array is referred to as a phased array.

In case of the CMUT transducers, which require a DC bias voltage for their activation, the transducer controller 18 can be coupled to control a DC bias control 45 for the transducer array. The DC bias control 45 sets DC bias voltage(s) that are applied to the CMUT transducer elements.

For each transducer element of the transducer array, analog ultrasound signals, typically referred to as channel data, enter the system by way of the reception channel. In the reception channel, partially beamformed signals are produced from the channel data by the microbeamformer 12 and are then passed to a main receive beamformer 20 where the partially beamformed signals from individual patches of transducers are combined into a fully beamformed signal, referred to as radio frequency (RF) data. The beamforming performed at each stage may be carried out as described above, or may include additional functions. For example, the main beamformer 20 may have 128 channels, each of which receives a partially beamformed signal from a patch of dozens or hundreds of transducer elements. In this way, the signals received by thousands of transducers of a transducer array can contribute efficiently to a single beamformed signal.

The beamformed reception signals are coupled to a signal processor 22. The signal processor 22 can process the received echo signals in various ways, such as: band-pass filtering; decimation; I and Q component separation; and harmonic signal separation, which acts to separate linear and nonlinear signals so as to enable the identification of nonlinear (higher harmonics of the fundamental frequency) echo signals returned from tissue and micro-bubbles. The signal processor may also perform additional signal enhancement such as speckle reduction, signal compounding, and noise elimination. The band-pass filter in the signal processor can be a tracking filter, with its pass band sliding from a higher frequency band to a lower frequency band as echo signals are received from increasing depths, thereby rejecting noise at higher frequencies from greater depths that is typically devoid of anatomical information.

The beamformers for transmission and for reception are implemented in different hardware and can have different functions. Of course, the receiver beamformer is designed to take into account the characteristics of the transmission beamformer. In FIG. 1 only the receiver beamformers 12, 20 are shown, for simplicity. In the complete system, there will also be a transmission chain with a transmission micro beamformer, and a main transmission beamformer.

The function of the micro beamformer 12 is to provide an initial combination of signals in order to decrease the number of analog signal paths. This is typically performed in the analog domain.

The final beamforming is done in the main beamformer 20 and is typically after digitization.

The transmission and reception channels use the same transducer array 6 which has a fixed frequency band. However, the bandwidth that the transmission pulses occupy can vary depending on the transmission beamforming used. The reception channel can capture the whole transducer bandwidth (which is the classic approach) or, by using bandpass processing, it can extract only the bandwidth that contains the desired information (e.g. the harmonics of the main harmonic).

The RF signals may then be coupled to a B mode (i.e. brightness mode, or 2D imaging mode) processor 26 and a Doppler processor 28. The B mode processor 26 performs amplitude detection on the received ultrasound signal for the imaging of structures in the body, such as organ tissue and blood vessels. In the case of line-by-line imaging, each line (beam) is represented by an associated RF signal, the amplitude of which is used to generate a brightness value to be assigned to a pixel in the B mode image. The exact location of the pixel within the image is determined by the location of the associated amplitude measurement along the RF signal and the line (beam) number of the RF signal. B mode images of such structures may be formed in the harmonic or fundamental image mode, or a combination of both as described in U.S. Pat. No. 6,283,919 (Roundhill et al.) and U.S. Pat. No. 6,458,083 (Jago et al.) The Doppler processor 28 processes temporally distinct signals arising from tissue movement and blood flow for the detection of moving substances, such as the flow of blood cells in the image field. The Doppler processor 28 typically includes a wall filter with parameters set to pass or reject echoes returned from selected types of materials in the body.

The structural and motion signals produced by the B mode and Doppler processors are coupled to a scan converter 32 and a multi-planar reformatter 44. The scan converter 32 arranges the echo signals in the spatial relationship from which they were received in a desired image format. In other words, the scan converter acts to convert the RF data from a cylindrical coordinate system to a Cartesian coordinate system appropriate for displaying an ultrasound image on an image display 40. In the case of B mode imaging, the brightness of pixel at a given coordinate is proportional to the amplitude of the RF signal received from that location. For instance, the scan converter may arrange the echo signal into a two dimensional (2D) sector-shaped format, or a pyramidal three dimensional (3D) image. The scan converter can overlay a B mode structural image with colors corresponding to motion at points in the image field, where the Doppler-estimated velocities to produce a given color. The combined B mode structural image and color Doppler image depicts the motion of tissue and blood flow within the structural image field. The multi-planar reformatter will convert echoes that are received from points in a common plane in a volumetric region of the body into an ultrasound image of that plane, as described in U.S. Pat. No. 6,443,896 (Detmer). A volume renderer 42 converts the echo signals of a 3D data set into a projected 3D image as viewed from a given reference point as described in U.S. Pat. No. 6,530,885 (Entrekin et al.).

The 2D or 3D images are coupled from the scan converter 32, multi-planar reformatter 44, and volume renderer 42 to an image processor 30 for further enhancement, buffering and temporary storage for display on an image display 40. The imaging processor may be adapted to remove certain imaging artifacts from the final ultrasound image, such as: acoustic shadowing, for example caused by a strong attenuator or refraction; posterior enhancement, for example caused by a weak attenuator; reverberation artifacts, for example where highly reflective tissue interfaces are located in close proximity; and so on. In addition, the image processor may be adapted to handle certain speckle reduction functions, in order to improve the contrast of the final ultrasound image.

In addition to being used for imaging, the blood flow values produced by the Doppler processor 28 and tissue structure information produced by the B mode processor 26 are coupled to a quantification processor 34. The quantification processor produces measures of different flow conditions such as the volume rate of blood flow in addition to structural measurements such as the sizes of organs and gestational age. The quantification processor may receive input from the user control panel 38, such as the point in the anatomy of an image where a measurement is to be made.

Output data from the quantification processor is coupled to a graphics processor 36 for the reproduction of measurement graphics and values with the image on the display 40, and for audio output from the display device 40. The graphics processor 36 can also generate graphic overlays for display with the ultrasound images. These graphic overlays can contain standard identifying information such as patient name, date and time of the image, imaging parameters, and the like. For these purposes the graphics processor receives input from the user interface 38, such as patient name. The user interface is also coupled to the transmit controller 18 to control the generation of ultrasound signals from the transducer array 6 and hence the images produced by the transducer array and the ultrasound system. The transmit control function of the controller 18 is only one of the functions performed. The controller 18 also takes account of the mode of operation (given by the user) and the corresponding required transmitter configuration and band-pass configuration in the receiver analog to digital converter. The controller 18 can be a state machine with fixed states.

The user interface is also coupled to the multi-planar reformatter 44 for selection and control of the planes of multiple multi-planar reformatted (MPR) images which may be used to perform quantified measures in the image field of the MPR images.

The methods described herein may be performed on a processing unit. Such a processing unit may be located within an ultrasound system, such as the system described above with reference to FIG. 1. For example, the image processor 30 described above may perform some, or all, of the method steps detailed below. Alternatively, the processing unit may be located in any suitable system, such as a monitoring system, that is adapted to receive an input relating to a subject.

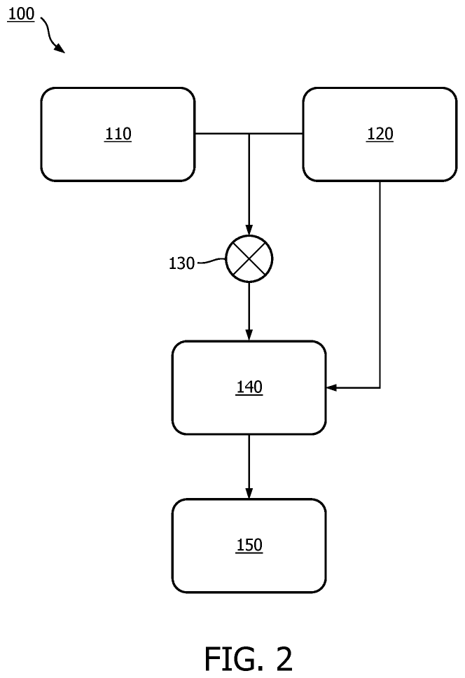

FIG. 2 shows a method 100 for obtaining a P-V loop from a subject. The P-V loop may be a P-V loop of the left ventricle of a subject.

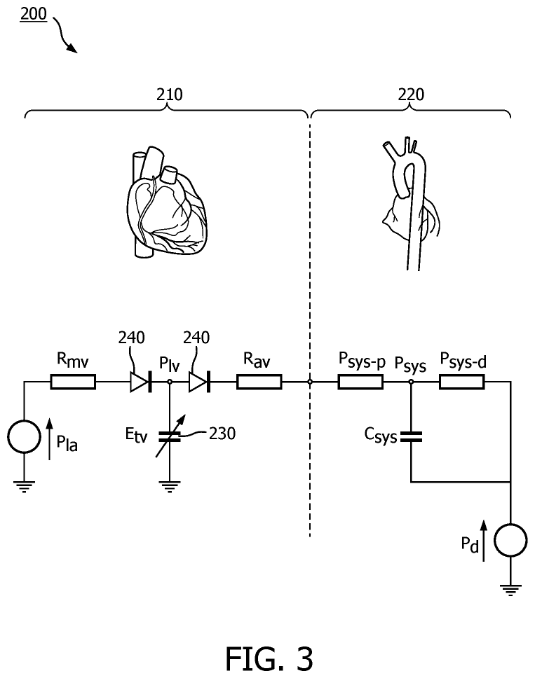

The method begins in step 110 with the obtaining of a numerical model of a cardiac system. The numerical model simulates the function of a given cardiac system, such as a left ventricle and systemic arteries or a heart. More specifically, the cardiac system may include the left heart and the systemic arteries. An example of a numerical model for a cardiac system is described below with reference to FIG. 3.

The numerical model may be constructed based on a variety of physical principles. For example, the numerical model may be based on any one or more of: a pressure-volume relationship; a stiffness of the cardiac system; conservation of energy; conservation of mass; and conservation of momentum. The numerical model may also include a systemic arterial model, representing the arterial system of a subject.

The incorporation of physical principles into the numerical model provides a framework within which data from various sources (such as: ultrasound data; peripheral blood pressure; clinical guidelines; machine learning estimates; and the like) can be fused together following physical principals such as the conservation of mass, momentum and energy.

Accordingly, a resultant estimate from the numerical model, such as a pressure estimate, may be made more consistent, particularly where inputs from different imaging modalities and/or different moments in time are used.

In step 120, physiological data is obtained from the subject in a non-invasive manner. The physiological data may be obtained from the subject in any suitable non-invasive manner.

For example, the physiological data may include: ultrasound data; blood pressure measurements; electrocardiogram data; electroencephalogram data; electromyography data; and respiratory data.

In particular, the ultrasound data may include one or more of: 2D ultrasound image data; 3D ultrasound image data; and Doppler ultrasound data. Further, the blood pressure may comprise one or more of: an arterial pressure waveform; and a cuff pressure value.

The availability of certain data types may vary according to the resources of a given situation. For example, in diagnostic applications high temporal resolution ultrasound volume and/or flow data may be available, with limited blood pressure data. Alternatively, in intensive care applications high temporal resolution blood pressure data may be available and ultrasound data may be unavailable or highly limited. In such situations, it may be possible to incorporate non-continuous volume data into the numerical model.

In an example, the physiological data includes ultrasound data of the left ventricle of a subject and arterial blood pressure. The ultrasound data may be segmented to extract a volume waveform of the left ventricle. In this way, the pressure information and volume information may be provided to the numerical model.

The volume waveform may be constructed from a least squares fit of an analytical waveform. The analytical waveform may, for example, comprise an aortic and a mitral flow wavform. The flow waveforms may be represented by one or more of: symmetric half-sine waveforms; asymmetric half-sine waveforms; filling waveforms with E- & A-waves with zero flow diastasis; filling waveforms with E- & A-waves with non-zero flow diastasis; and filling waveforms with E- & A-waves with no diastasis. The E-wave refers to the early filling of the left ventricle, before the left atrium contracts; whereas, the A-wave refers to the filling of the left ventricle during artial contraction.

The analytical fit provides a more robust method for reconstructing a volume waveform by using constraints which preserve physiological events (such as isovolumetric phases) which may be missed from ultrasound data with a limited frame rate.

The analytical volume waveform may be further adjusted based on additional physiological data, such as ECG data or Doppler ultrasound data.

Further, the analytical volume waveform may also be simplified, as volume segmentations are not required from all ultrasound image frames. The volume fitting may be performed using only an end systolic volume, an end diastolic volume and their associated timings.

In step 130, the numerical model is updated based on the physiological data.

In other words, patient specific data may be used to tune the numerical model to an individual user. By using patient-specific inputs (such as ultrasound volume segmentation taken from the ultrasound data or peripheral pressure measurements), the numerical model can be personalized to each patient, which in turn provides a patient-specific estimate of a cardiac function, for example ventricular pressure.

The updating of the numerical model may include identifying a model parameter based on the physiological data and providing the model parameter to the numerical model.

For example, the model parameter may include one or more of: a systemic circulation parameter; a filling parameter; an ejection parameter; and a stiffness parameter.

For example, an arterial blood pressure measurement may be used to tune the parameters of a system arterial model. The arterial blood pressure measurement may comprise one or more of a maximum, a minimum and a mean blood pressure value. The arterial blood pressure measurement may be used to adjust a resistance and a compliance of the systemic system in the numerical model.

In order to derive a ventricular pressure measurement, an estimate of the pressure gradient over the aortic valve is required. The pressure gradient over the aortic valve may be estimated using Doppler ultrasound measurements. In the simplest implementation, the pressure gradient over the aortic valve may be assumed to be 0.

The calibration of the numerical model may be further improved using a full blood pressure waveform. In this way, the numerical model may be further personalized to the subject, for example by including pressure decay constants in the simulation of the cardiac system. A full blood pressure waveform may be determined non-invasively, for example, by way of applanation tonometry or vessel diameter measurements by ultrasound imaging.

The blood pressure information used to calibrate the numerical model may be obtained from a peripheral artery, such as a brachial or radial aretry. Measurements of pressures in such locations are amplified with respect to central aortic pressure values due to variations in vessel diameter and stiffness. This may be corrected for through a variety of methods, such as a mathematical transfer function or applying a scaling of minimum/maximum values based on population data from different demographics (which may include sex, age, known blood pressure conditions and the like).

In the numerical model, the model parameters may be identified using a multi-step approach. For example, parameters that represent the systemic circulation may be identified first (i.e. the parameters associated with the afterload of the cardiac system), before the parameters representing the ejection and filling of the heart are identified. These patient-specific parameters may be estimated using a mixture of techniques such as physiology based rules, direct optimization methods, sequential filtering methods and the like.

In step 140, the physiological data is provided to the updated numerical model.

Thus, the numerical model simulates the cardiac system in a way that mimics the behavior of the subject. Accordingly, the numerical model may provide for a means of interpreting the physiological data taken from the subject within the context of an accurate representation of the subject's cardiac system.

For example, the analytical volume waveform described above may used as an input to the numerical model, for example the systemic arterial model, which has been tuned according to an arterial pressure measurement, to reconstruct the systemic part of the P-V loop.

The incorporation of physical principles into the numerical model provides for the automatic alignment of the pressure measurements with the cardiac ultrasound measurements.

In step 150, a physiological measure, and in particular a P-V loop, is derived based on an output of the updated numerical model.

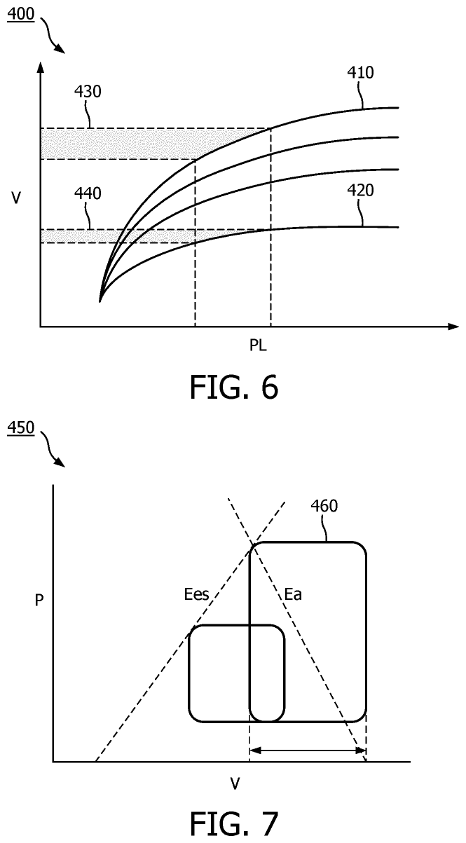

For example, the physiological measure may comprise, in addition to a pressure-volume loop: a pressure-strain relationship; and a fluid responsiveness, which is discussed further below with reference to FIG. 6.

The P-V loop may then be displayed to a user, such as a care taker or a medical professional associated with the subject.

In the example described above, where the physiological data only includes ultrasound data of the left ventricle of a subject and arterial blood pressure, the P-V loop only conveys systolic information. In other words, the resulting P-V loop is a partial P-V loop relating to the systolic behavior of the left ventricle.

The P-V loop may be completed using diastolic pressure information. The diastolic filling pressure input may be obtained non-invasively using a population based ultrasound pressure surrogate such as an E/E′ ratio, which is the ratio between early mitral inflow velocity and mitral annular early diastolic velocity.

The correlation of the surrogate pressure measurement with the corresponding time in the analytical volume waveform provides a linearized estimate of the pressure-volume relationship during filling. This enables the extrapolation of the pressure during diastolic filling and the completion of the P-V loop.

FIG. 3 shows a schematic representation of a numerical model 200 of a cardiac system, namely, the left heart 210 and the aorta 220.

In this example, a simplified 0D approach to modeling blood flow during the heart cycle is represented. However, it is also possible to combine 1D/3D modeling approaches within the described framework and it is further possible to include a model of a complete circulatory system.

For example, in a diagnostic setting, a numerical model may be required to estimate a ventricular pressure in the left ventricle of a subject. FIG. 3 illustrates an example of such a numerical model 200, represented as an electronic circuit.

In the model shown in FIG. 3, the voltage represents the blood pressure and the current represents the blood flow. In this approach the different compartments of the arterial system (such as the atria, ventricles, large arteries, and so on) are grouped together into electrical components which are related to hemodynamic analogues, such as resistance and capacitance.

Beginning with the left heart 210, the source (Pₗₐ) will charge the variable capacitor 230, mimicking the left atrium pumping blood to the left ventricle. The left atrium will fill the left ventricle (variable capacitor) to its passive limit.

The capacitance of the variable capacitor 230 represents the stiffness of the ventricle, i.e. the muscular contraction. The volume is a state variable that may be derived from the physiological data of the user.

Eₗᵥ refers to the elastance of the left ventricle. This relates the ventricular volume to the pressure within the left ventricle. Although it is a measure of the stiffness (i.e. muscular contraction of the ventricle), it is not strictly a material stiffness. This relationship has been experimentally measured from simultaneous ventricular pressure and volume waveforms.

The charge travels along the circuit, with the diodes 240 acting as valves to define a direction of flow. The blood (charge) then moves into the aorta 220 and enters the systemic circulation system.

The resistance terms (mitral valve resistance, Rₘᵥ, aortic valve resistance, Rₐᵥ, proximal systemic resistance, R_{sys=p}, distal systemic resistance, R_{sys=d}) represent the resistance of the blood vessels and valves to the blood flow and are directly related to the pressure in a given area. C_{sys} represents systemic compliance.

In this example, the model parameters are represented using electrical analogues. However, such models do follow physical principle such as conservation of mass, for example the blood flow (current) into each node of the model is conserved. Furthermore, the electrical analogue can be derived from the linearization of the mass and momentum conservation equation for blood flow in deformable vessels.

Combining the pressure estimate of the model with measurements of ventricle volume (for example from the ultrasound data obtained from the subject) it is possible to construct pressure-volume loops for the subject.

It should be noted that the example shown in FIG. 3 is only one of many possible models of the left heart. The different components of the model may be interchanged dependent upon the specific application. For example the model may be adapted to include a dynamic left atrium, a regurgitant mitral valve and the like. As described above, the numerical model may also include a systemic arterial model.

In this example, it is assumed that ultrasound volume information is available. However, the availability of such information depends upon the specific application area, such as in patient monitoring applications the availability of ultrasound volume data is more limited than in diagnostic applications. The model may be adapted to incorporate additional ultrasound data for specific applications, such as Doppler waveforms in heart diagnosis; however, this additional data must be routinely collected.

The numerical model described above may be integrated into an ultrasound analysis platform. This may enable the model to utilize the patient-specific segmentation of the left heart.

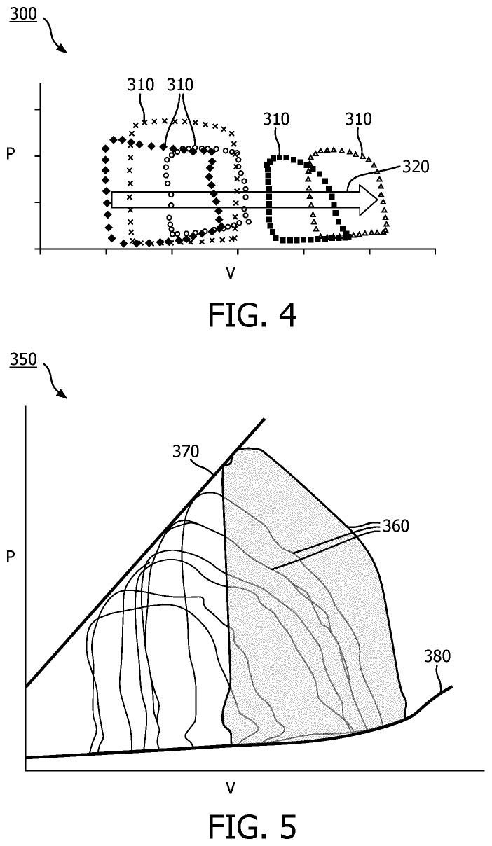

FIG. 4 shows a graph 300 of pressure, P, against volume, V.

The simultaneous variations in pressure and volume with the heart can be utilized to construct a pressure-volume loop 310 of the left ventricle.

The range of ventricular pressures and volumes provides insight into the pumping performance of the heart, for example the energy expended with each heartbeat or ventriculo-arterial efficiency. This enables a characterization of heart failure, wherein a heart failure trend 320 may be identified as described in D. R. Warriner et al., “Closing the loop: Modeling of heart failure progression from health to end-stage using a meta-analysis of left ventricular pressure-volume loops,” PLoS One, vol. 9, no. 12, pp. 1-19, 2014.

FIG. 5 shows a graph 350 of pressure, P, against volume, V, with a series of pressure-volume loops 360.

In addition to the uses described above, pressure-volume loops 360 may also provide information on the ability of the heart to respond to different conditions, such as exercise, stress and drugs. By analyzing multiple pressure-volume loops from different heartbeats, key physiological parameters such as the end-systolic pressure-volume relationship 370 and end-diastolic pressure-volume relationship 380 can be determined as discussed in D. Burkhoff, “Assessment of systolic and diastolic ventricular properties via pressure-volume analysis: a guide for clinical, translational, and basic researchers,” AJP Hear. Circ. Physiol., vol. 289, no. 2, pp. H501-H512, 2005. Such metrics reveal the ability, or inability, of the heart to alter its pumping and filling performance.

As described above, current integrated solutions for pressure-volume loop reconstruction are invasive (for example, the Millar Inca system), with dual catheter systems for simultaneous recordings of pressure and volume. The measurement of volume is typically performed using a conductance catheter technique; however, such devices are reported to have similar accuracy to non-invasive ultrasound methods as demonstrated in C.-H. Chen et al., “Comparison of Continuous Left Ventricular Volumes by Transthoracic Two-Dimensional Digital Echo Quantification with Simultaneous Conductance Catheter Measurements in Patients with Cardiac Diseases,” Am. J. Cardiol., vol. 80, no. 6, pp. 756-761, September 1997.

Accordingly, the non-invasive measurements may be provided to the numerical model described above without a loss of accuracy.

Alongside ventricular volume (which may be acquired by way of the ultrasound data), the generation of ventricular pressure during contraction of the heart may also be correlated to other metrics, such as strain. Also related to changes in volume, strain is a measure of the dimensional changes occurring during contraction. As strain can be measured locally, a pressure-strain plot can be used to characterize the contractile performance of different regions of the ventricle as discussed in E. Samset, “Evaluation of segmental myocardial work in the left ventricle”.

In other words, the strain may be measured and form part of the physiological data acquired from the subject. The strain data may then be provided to the numerical model in order to derive a measure of the cardiac function of the subject. As discussed earlier, the estimation of pressure is personalized to the subject (for example via a model based approximation or mathematical transformation).

The numerical model described above may also be used to perform a real-time assessment of a fluid responsiveness of the subject.

For critically ill patients with inadequate tissue perfusion (for example, those who have experienced shock) the administration of fluids is typically the first step to restoring perfusion. The addition of extra fluid increases the preload on the heart, which aims to increase stroke volume/cardiac output, thereby restoring adequate perfusion.

FIG. 6 shows a graph 400 of preload, PL, against volume, V.

The graph contains a number of plots, wherein plot 410 indicates the normal systolic function and plot 420 indicates poor systolic function.

The relative increase in stroke volume is dependent upon the current preload of the heart. The relationship between preload and stroke volume, also known as the Frank-Starling mechanism, is non-linear as illustrated in the graph 400. At lower levels of preload, the heart is able to generate larger increases in stroke volume when compared to higher levels of preload, as the contractile force of the heart reaches a maximum.

Furthermore, the functional state of the heart itself plays a role in stroke volume generation. For hearts with decreased systolic function, the relative increase in stroke volume is significantly smaller than a heart with normal systolic function for the same change in preload as shown by areas 430 and 440, wherein area 430 represents the relative increase in stroke volume for a heart with decreased systolic function and area 440 represents the relative increase in stroke volume for a heart with normal systolic function.

The stroke volume of the cardiac system may be determined from the ultrasound data as described above.

This ability to respond to additional fluids by increasing stroke volume is typically referred to as fluid responsiveness and may be derived from the numerical model of the cardiac system. Clinical studies have shown that only approximately 50% of critically ill patients are display adequate fluid responsiveness as demonstrated in P. E. Marik, X. Monnet, and J.-L. Teboul, “Hemodynamic parameters to guide fluid therapy,” Ann. Intensive Care, vol. 1, no. 1, p. 1, 2011.

Thus, the ability of the numerical model to assess fluid responsiveness in real time may provide a tool to classify subject who will positively benefit from fluid administration.

The operational point of a subject on the Frank-Starling curve can be directly assessed by pressure-volume loop analysis. By reconstructing the pressure-volume loop, as described above, on a beat-by-beat basis, the relationship between preload and stroke volume can be directly determined.

The numerical model described above may also be used to perform a real-time detection of septic shock in the subject.

Septic shock is a hypotensive (low blood pressure) state that leads to ischemia, organ dysfunction and ultimately death. As septic shock affects both the heart and the circulation, analyzing the variations in the ventriculo-arterial coupling, as simulated by the numerical model, provides an approach to detecting septic shock.

FIG. 7 shows a graph 450 of volume, V, against pressure, P, with a plot 460 representing a pressure-volume loop derived form a subject.

One measure of the ventriculo-arterial coupling is the ratio of the end systolic elastance, Ees, to the arterial elastance, Ea. In humans, the normal ratio of Ees/Ea is approximately 1.0, signifying that the heart is operating at optimal efficiency. However, in cases of septic shock F. Guarracino, B. Ferro, A. Morelli, P. Bertini, R. Baldassarri, and M. R. Pinsky, “Ventriculoarterial decoupling in human septic shock,” Crit. Care, vol. 18, no. 2, p. R80, 2014 demonstrated that this ratio is elevated to over 1.36. These metrics are determined from the pressure-volume loop as illustrated in FIG. 7 and may be derived from the numerical model described above.

The current recommendations for hemodynamic management of septic shock include monitoring of arterial pressure by cannulation and assessment of filling pressures by catheterization or echocardiography. However, such information is also included within a pressure-volume loop. Thus, providing a real-time estimate of the pressure-volume loop will provide metrics currently used to assess septic shock—alongside another potential metrics of septic shock and the ventriculo-arterial coupling. Furthermore, real-time pressure-volume loops as derived from the numerical model will enable the effect of treatment by vasopressors to be observed, providing a tool for clinicians to detect and treat septic shock.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer-implemented method for obtaining a physiological measure from a subject, the method comprising:
obtaining a numerical model of a cardiac system;
obtaining, in a non-invasive manner, physiological data from the subject;
updating the numerical model based on the physiological data;
providing the physiological data to the updated numerical model, the physiological data comprising strain data; and
deriving a physiological measure based on an output of the updated numerical model, the physiological measure comprising a pressure-volume, P-V, loop, and a pressure-strain relationship.

2. A method as claimed in claim 1, wherein the physiological data comprises one or more of:
ultrasound data;
electrocardiogram data; and
a blood pressure.

3. The method as claimed in claim 2, wherein the ultrasound data comprises one or more of:
2D ultrasound image data;
3D ultrasound image data; and
Doppler ultrasound data.

4. The method as claimed in claim 2, wherein the blood pressure comprises one or more of:
an arterial pressure waveform; and
a cuff pressure value.

5. The method as claimed in claim 1, wherein the numerical model is based on:
a pressure-volume relationship;
a stiffness of the cardiac system;
conservation of energy;
conservation of mass; and
conservation of momentum.

6. The method as claimed in claim 1, wherein the cardiac system comprises a left ventricle and systemic arteries.

7. The method as claimed in claim 1, wherein the cardiac system comprises a heart.

8. The method as claimed in claim 1, wherein the updating of the numerical model comprises:
identifying a model parameter based on the physiological data; and
providing the model parameter to the numerical model.

9. The method as claimed in claim 8, wherein the model parameter comprises one or more of:
a systemic circulation parameter;
a filling parameter;
an ejection parameter; and
a stiffness parameter.

10. (canceled)

11. The method as claimed in claim 1, wherein the physiological measure further comprises a fluid responsiveness.

12. The method as claimed in claim 1, wherein the method further comprises displaying the physiological measure to a user.

13. A computer program comprising computer program code means which is adapted, when said computer program is run on a computer, to implement the method of claim 1.

14. A processing unit for obtaining a physiological measure from a subject, wherein the processing unit is adapted to:
obtain a numerical model of a cardiac system;
update the numerical model based on the physiological data;
provide the physiological data to the updated numerical model, the physiological data comprising strain data; and
derive a physiological measure based on an output of the updated numerical model, wherein the physiological measure comprises a pressure-volume, P-V, loop, and a pressure-strain relationship.

15. A system for obtaining a physiological measure from a subject, the system comprising:
a physiological sensor adapted to obtain physiological data from the subject in a non-invasive manner, wherein the physiological sensor comprises one or more of:
an ultrasound transducer, wherein the physiological data comprises ultrasound data;
an electrocardiogram sensor, wherein the physiological data comprises electrocardiogram data; and
a blood pressure measurement device, wherein the physiological data comprises blood pressure data; and
a processor as claimed in claim 14.
